(19) United States
(12) Patent Application Publication
(10) Pub. No.: US 2015/0272447 A1
(43) Pub. Date: Oct. 1, 2015

(54) PACIFIER SENSITIVE TO INTERNAL AND EXTERNAL TEMPERATURES

(71) Applicant: SUCETTE INC., Powder Springs, GA (US)

(72) Inventors: Rachel Kathlyn Ford, Powder Springs, GA (US); Frederick Grimm, North Augusta, SC (US); William Harrison McAllister, Charlotte, NC (US); Esteban David Ongini Smoljo, Surfside, FL (US); Kaitlyn Elizabeth Wolfe, Bradenton, FL (US)

(21) Appl. No.: 14/668,061

(22) Filed: Mar. 25, 2015

Related U.S. Application Data
(60) Provisional application No. 61/969,826, filed on Mar. 25, 2014.

(51) Int. Cl. A61B 5/01; A61J 17/00

(52) U.S. Cl. CPC A61B 5/01; A61J 17/00; A61J 2017/003

(57) ABSTRACT

The disclosed technology includes a temperature-sensitive pacifier for providing early warnings of dangerous temperature-related conditions, such as fever or a risk of heat stroke. The temperature-sensitive pacifier may integrate a thermochromic material into the nipple, neck, and base portions of the pacifier to provide a visual indication of an oral temperature condition, and separately provide a visual indication of an environmental temperature condition.

### CROSS-REFERENCE TO RELATED APPLICATIONS

This application claims priority to, and the benefit under 35 U.S.C. §119(e) of, U.S. Provisional Patent Application No. 61/969,826, filed 25 Mar. 2014, the entire contents and substance of which are hereby incorporated by reference as if fully set forth below.

### TECHNICAL FIELD

Aspects of the present disclosure relate to a child's pacifier. More particularly, aspects of this disclosure relate to a pacifier that may provide visual indications of internal and external temperature conditions to provide an early warning of dangerous conditions such as fever and heat stroke.

### BACKGROUND

Many parents are highly sensitive to the health concerns of their children. Babies and infants who are not yet able to communicate and who may be more vulnerable to potential health problems are of particular concern. Fevers are a symptom of a variety of possible health problems. The presence of a fever in an infant or newborn may indicate a serious or possibly life-threatening illness. Further, if an infant is subjected to an environment that is too hot, the infant may suffer from heat stroke. Babies and infants are particularly at risk from heat stroke, which can also be potentially life-threatening. As such, there is a persistent need for early warning indications of potentially dangerous conditions such as fever and heat stroke.

Mercurial or electronic thermometers may be used to measure the ambient temperature of the environment, as well as a child's body temperature, by placing the thermometer in the child's mouth. But using a thermometer is impractical for detecting early warning signs of overheating as a parent would need to take constant readings from the thermometer to recognize any early warning signs. Therefore, a more passive means of providing an early warning of overheating is preferably desired. Thus, there is a need for a pacifier that may provide an early warning of dangerous conditions such as fever or a risk of heat stroke, in a manner that may be passively monitored by a parent.

### SUMMARY

Some or all of the above deficiencies may be addressed by certain embodiments of the disclosed technology.

According to an example embodiment, a temperature-sensitive pacifier is provided. The temperature-sensitive pacifier may comprise a nipple portion coupled to a neck portion, a base portion coupled to the neck portion, and a guard portion substantially encapsulating the base portion. In some embodiments, the nipple portion, neck portion, and base portion may comprise a single member. The guard portion may be comprised of a substantially transparent material. The guard portion may be comprised of a material having a thermal conductivity of at least 0.2 W/mΩ·K. In some embodiments a temperature-sensitive pacifier may further comprise a handle member coupled to the guard portion.

According to example embodiments, the nipple portion may be comprised of a first thermochromic material configured to provide a visual indication of a body temperature condition and the base portion may be comprised of a second thermochromic material configured to provide a visual indication of an environmental temperature condition. In some embodiments the first thermochromic material and/or the second thermochromic material may comprise thermochromic liquid crystals or leucodyes. In some embodiments, the leucodyes may comprise two or more leucodye layers, each leucodye layer having a different color and a different set point.

According to some embodiments, the nipple portion may provide a visual indication of a body temperature condition in response to absorbing heat from a mouth of a user. For example, the nipple portion may change colors, transition from a first color to a second color, or become transparent when a user has a body temperature indicative of a fever or at an oral temperature of 99.7 degrees Fahrenheit.

According to some embodiments, the base portion may provide a visual indication of an environmental temperature condition in response to absorbing heat from a surrounding environment. For example, the base portion may change colors, transition from a first color to a second color, or become transparent when the surrounding environment poses a risk of heat stroke or has a temperature of 99.7 degrees Fahrenheit.

According to another example embodiment, a temperature-sensitive pacifier may further comprise a processor, at least one sensor in communication with the processor, and a wireless transmitter in communication with the processor. The sensor may be configured to measure at least one temperature of the nipple portion or the base portion of the pacifier. The wireless transmitter may be configured to wirelessly transmit data indicative of the at least one temperature to a computing device.

According to another example embodiment, the nipple portion of the temperature-sensitive pacifier may comprise an upper branch member and a lower branch member.

Other embodiments, features, and aspects of the disclosed technology are described in detail herein and are considered a part of the claimed disclosed technology. Other embodiments, features, and aspects can be understood with reference to the following detailed description, accompanying drawings, and claims.

### BRIEF DESCRIPTION OF DRAWINGS

Reference will now be made to the accompanying figures and flow diagrams, which are not necessarily drawn to scale, and wherein:

FIG. 1A is a front perspective view of a temperature-sensitive pacifier, in accordance with an exemplary embodiment of the presently disclosed subject matter.

FIG. 1B is a side view of a temperature-sensitive pacifier, in accordance with an exemplary embodiment of the presently disclosed subject matter.

FIG. 2A is a front perspective view of a temperature-sensitive pacifier, in accordance with an exemplary embodiment of the presently disclosed subject matter.

FIG. 2B is a rear perspective view of a temperature-sensitive pacifier, in accordance with an exemplary embodiment of the presently disclosed subject matter.

FIG. 2C is a front perspective view of a temperature-sensitive pacifier, in accordance with an exemplary embodiment of the presently disclosed subject matter.

FIG. 2D is an exploded view exemplary embodiment of a temperature-sensitive pacifier, in accordance with an exemplary embodiment of the presently disclosed subject matter.

FIG. 2E is a rear perspective view of a temperature-sensitive pacifier, in accordance with an exemplary embodiment of the presently disclosed subject matter.

FIG. 2F is an exploded view of a temperature-sensitive pacifier, in accordance with an exemplary embodiment of the presently disclosed subject matter.

FIG. 2G is a side view of a temperature-sensitive pacifier, in accordance with an exemplary embodiment of the presently disclosed subject matter.

FIG. 2H is an exploded side view of a temperature-sensitive pacifier, in accordance with an exemplary embodiment of the presently disclosed subject matter.

FIG. 2I is a bottom view of an exemplary embodiment of a temperature-sensitive pacifier, in accordance with an exemplary embodiment of the presently disclosed subject matter.

FIG. 2J is an exploded top view of a temperature-sensitive pacifier, in accordance with an exemplary embodiment of the presently disclosed subject matter.

FIG. 2K is a front view of an exemplary embodiment of a temperature-sensitive pacifier, in accordance with an exemplary embodiment of the presently disclosed subject matter.

FIG. 2L is a rear view of a temperature-sensitive pacifier, in accordance with an exemplary embodiment of the presently disclosed subject matter.

FIG. 2M is an alternate top view of a temperature-sensitive pacifier, in accordance with an exemplary embodiment of the presently disclosed subject matter.

FIG. 3A is a front perspective view of a temperature-sensitive pacifier, in accordance with an exemplary embodiment of the presently disclosed subject matter.

FIG. 3B is a side view of a temperature-sensitive pacifier, in accordance with an exemplary embodiment of the presently disclosed subject matter.

FIG. 4 is a side view of an exemplary embodiment of a temperature-sensitive pacifier, in accordance with an exemplary embodiment of the presently disclosed subject matter.

### DETAILED DESCRIPTION

The present disclosure can be understood more readily by reference to the following detailed description of exemplary embodiments and the examples included herein. Before the exemplary embodiments of the devices and methods according to the present disclosure are disclosed and described, it is to be understood that embodiments are not limited to those described within this disclosure. Numerous modifications and variations therein will be apparent to those skilled in the art and remain within the scope of the disclosure. It is also to be understood that the terminology used herein is for the purpose of describing specific embodiments only and is not intended to be limiting. Some embodiments of the disclosed technology will be described more fully hereinafter with reference to the accompanying drawings. This disclosed technology may, however, be embodied in many different forms and should not be construed as limited to the embodiments set forth therein.

In the following description, numerous specific details are set forth. But it is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Unless otherwise noted, the terms used herein are to be understood according to conventional usage by those of ordinary skill in the relevant art. In addition to any definitions of terms provided below, it is to be understood that as used in the specification and in the claims, “a” or “an” can mean one or more, depending upon the context in which it is used. Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

Unless otherwise specified, the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

Also, in describing the exemplary embodiments, terminology will be resorted to for the sake of clarity. It is intended that each term contemplates its broadest meaning as understood by those skilled in the art and includes all technical equivalents that operate in a similar manner to accomplish a similar purpose.

To facilitate an understanding of the principles and features of the embodiments of the present disclosure, exemplary embodiments are explained hereinafter with reference to their implementation in an illustrative embodiment. Such illustrative embodiments are not, however, intended to be limiting.

The materials described hereinafter as making up the various elements of the embodiments of the present disclosure are intended to be illustrative and not restrictive. Many suitable materials that would perform the same or a similar function as the materials described herein are intended to be embraced within the scope of the exemplary embodiments. Such other materials not described herein can include, but are not limited to, materials that are developed after the time of the development of the invention, for example.

Embodiments of the disclosed technology include a pacifier sensitive to internal and external temperatures (a “temperature-sensitive pacifier”) for providing an indication of an internal temperature condition and an indication of an external temperature condition. An internal temperature condition may represent a temperature condition present inside a user's mouth. A temperature condition present inside a user's mouth may provide an indication of a user's body temperature. An external temperature condition may represent a temperature condition of the environment surrounding the user, such as the ambient temperature of a room. Various embodiments of the disclosed technology may provide a temperature-sensitive pacifier wherein portions of the pacifier may separately change or lose color in response to internal and external temperature conditions. In some embodiments, a portion of the pacifier may change or lose color in response to an internal temperature condition indicative of the user having a fever (i.e., a body temperature above the normal range). In some embodiments, a portion of the pacifier may change or lose color in response to an external temperature condition indicative of the environment having a temperature that poses a risk of heat stroke.

Throughout this disclosure, certain embodiments are described in exemplary fashion in relation to being used by a baby or infant. But embodiments of the disclosed technology are not so limited. In some embodiments, the disclosed technique may be effective when used by a child, a teenager, an adult, or an animal. Further, while generally referred to as a “temperature-sensitive pacifier,” exemplary embodiments may also be referred to simply as a “pacifier.”

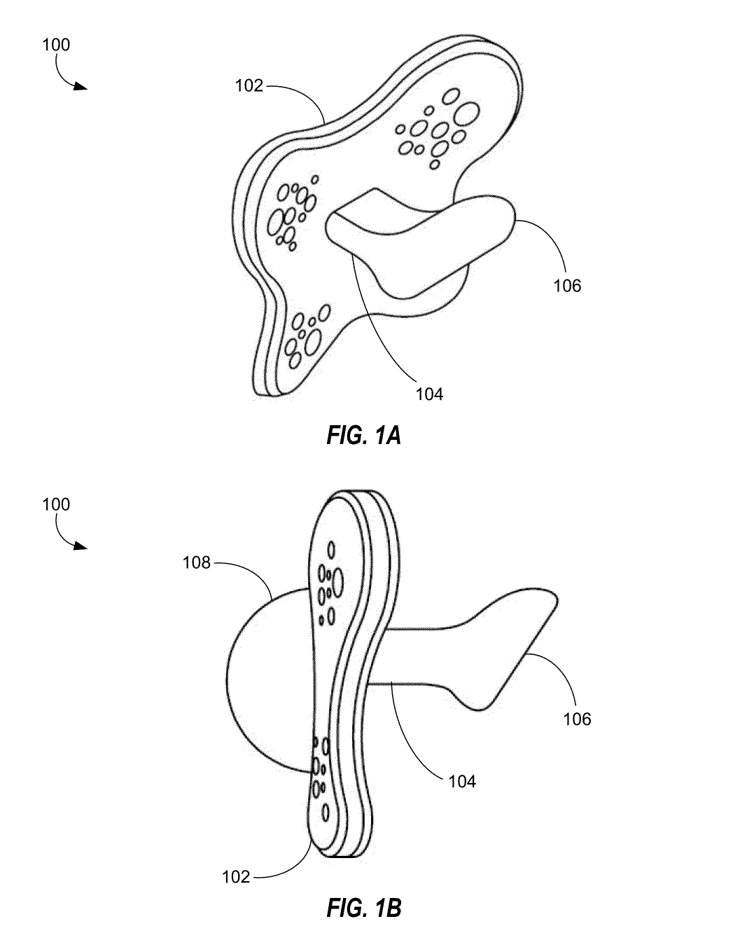

FIGS. 1A-1B illustrate an exemplary embodiment of a temperature-sensitive pacifier 100. In various embodiments, a temperature-sensitive pacifier may comprise a guard portion 102, a neck portion 104, a nipple portion 106 and a handle member 108. A temperature-sensitive pacifier may be used in the standard manner that pacifiers are generally used, wherein the nipple portion 106 and neck portion 104 are configured to be placed in an infant's mouth such that the infant can suck on the nipple portion 106. The guard portion 102 may act to prevent the infant from attempting to swallow the pacifier 100 by creating a resistive force when pressed against the infant's face. The handle member 108 may be used to pull the temperature-sensitive pacifier 100 out of an infant's mouth. Many shapes, sizes, and designs of pacifiers are known. The temperature-sensitive pacifier 100 illustrated in FIG. 1 is not intended to limit the design of the present disclosure, as it is contemplated that the present technology could be embodied in a variety of guard portions 102, neck portions 104, nipple portions 106 or handle members 108 of different shapes, sizes, and designs.

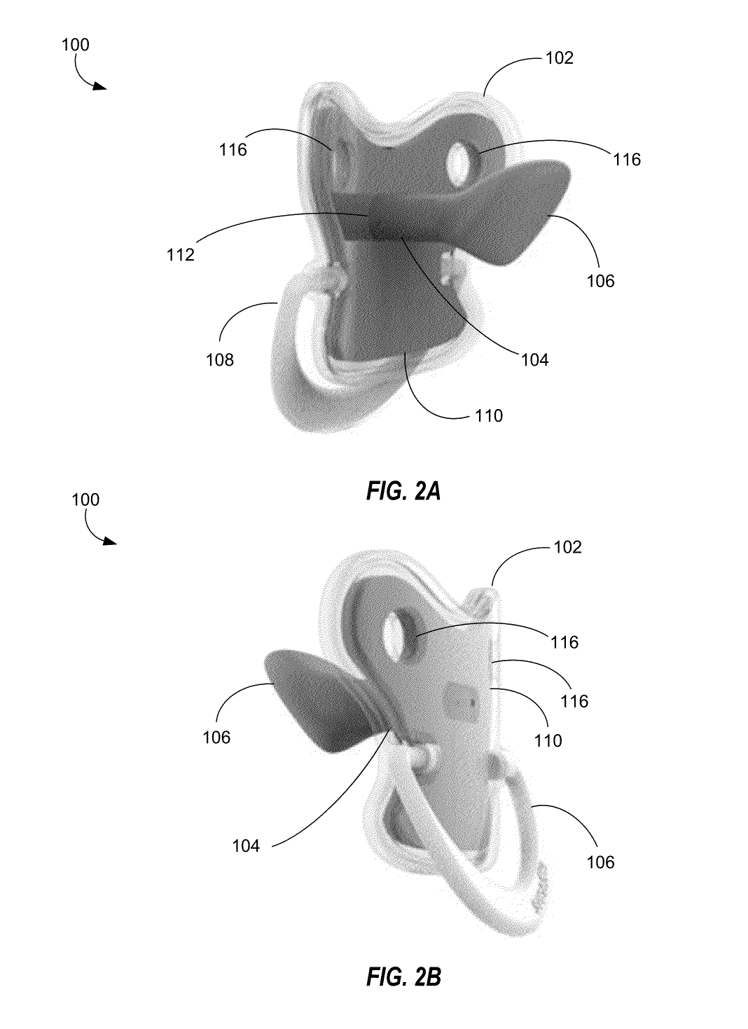

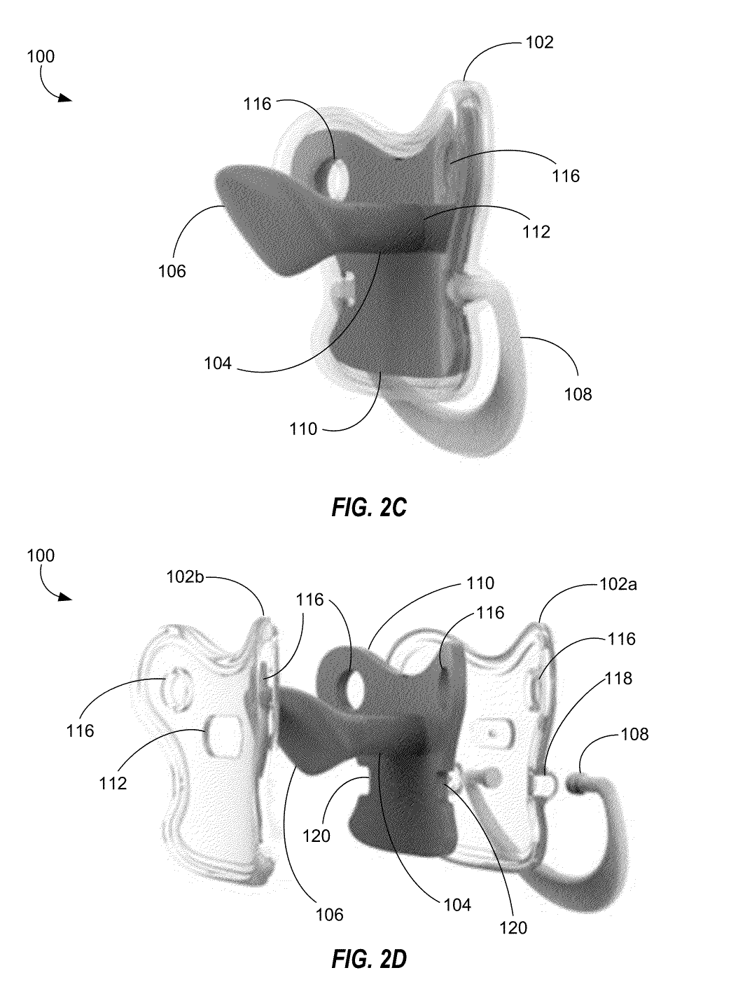

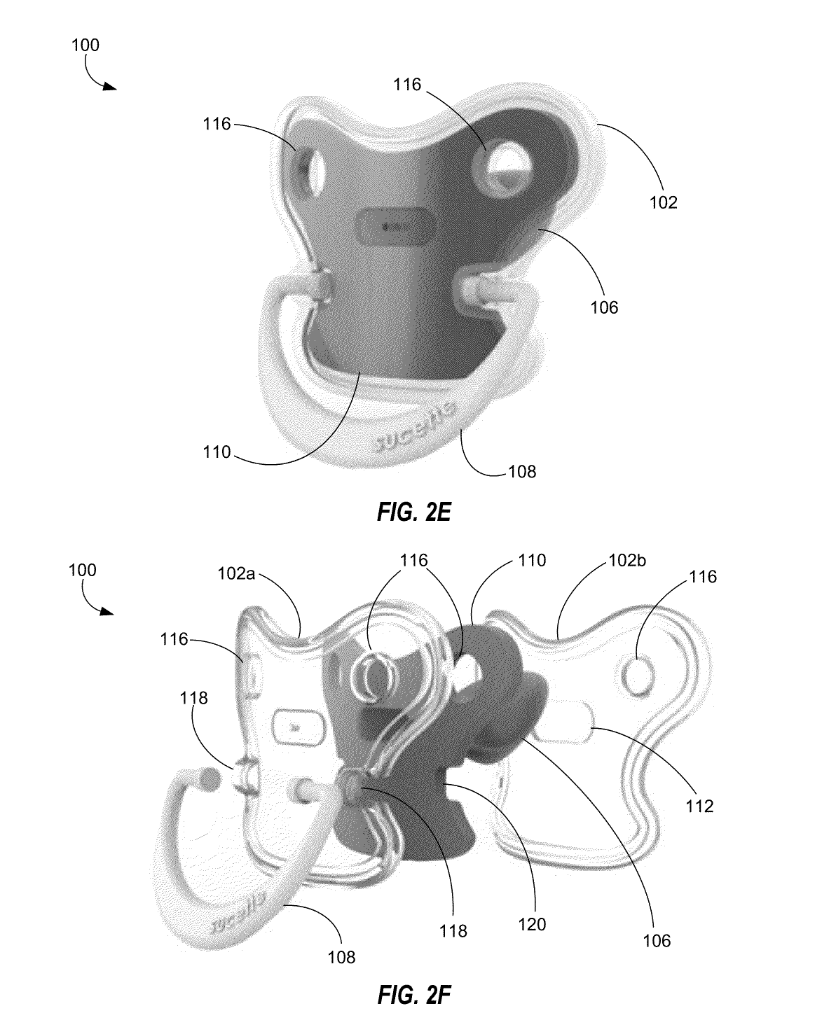

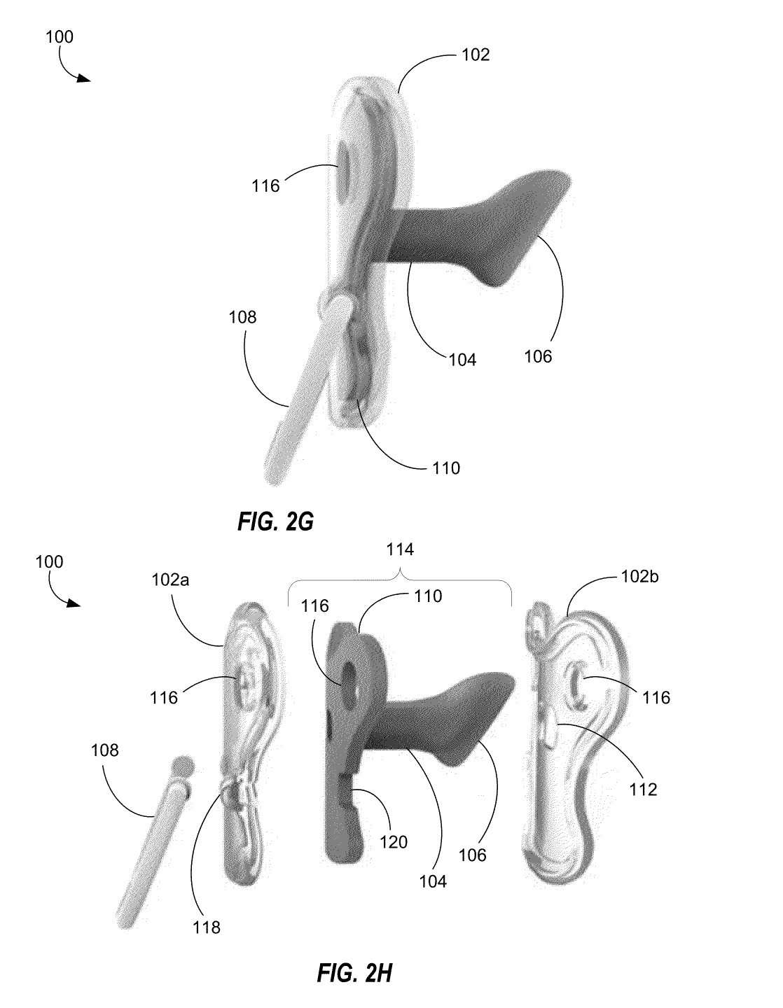

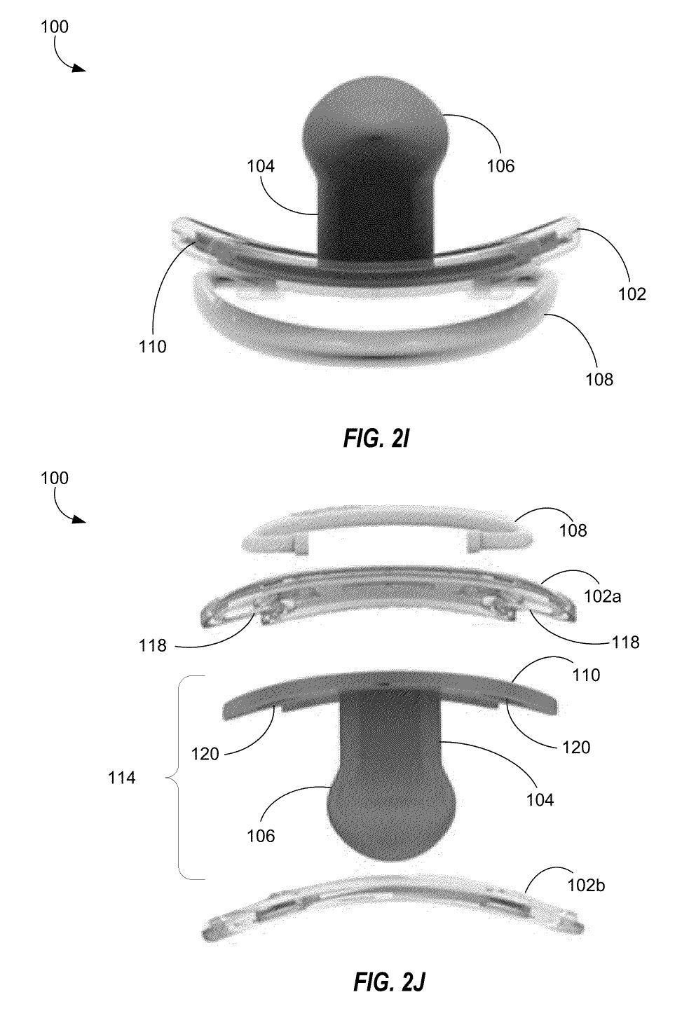

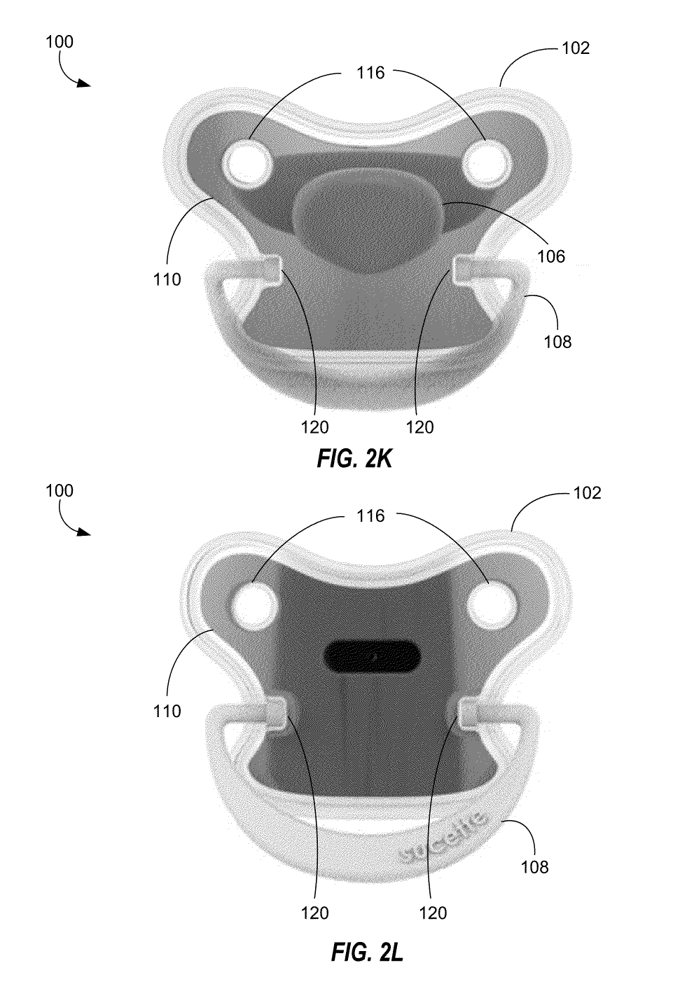

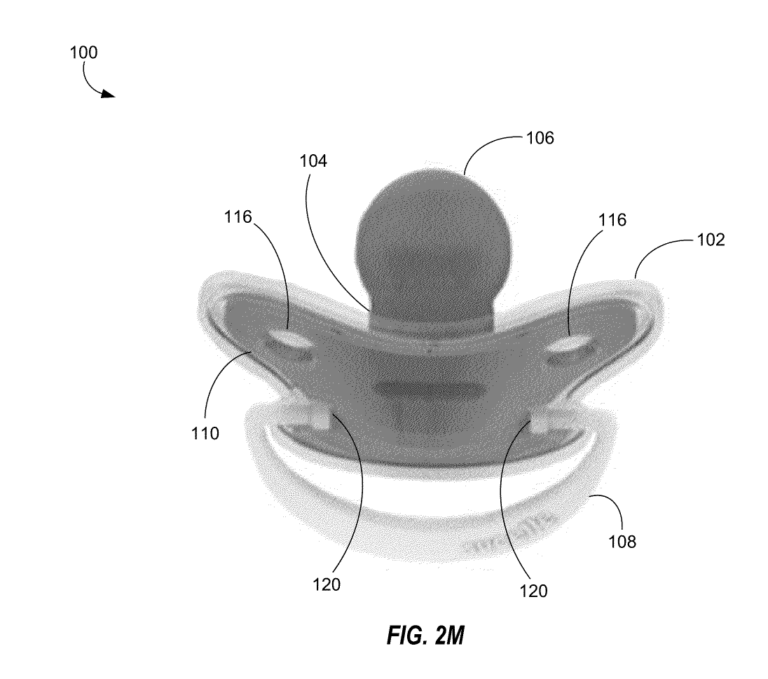

FIGS. 2A-2M illustrate an exemplary embodiment of a temperature-sensitive pacifier 100, additionally showing a base portion 110 that is coupled to the neck portion 104 and substantially encapsulated by the guard portion 102. In some embodiments, the guard portion 102 may create a seal around the base portion 110 such that no surface of the base portion 110 is directly exposed to the external environment. FIGS. 2D, 2F, 2H, and 2J are exploded views of an embodiment of a temperature-sensitive pacifier 100 showing that the guard portion 102 may comprise a rear guard portion 102a and a front guard portion 102b. In various embodiments, the front guard portion 102b may contain an aperture 112. According to some embodiments, the neck portion 104 may extend away from the base portion 110 through the aperture 112. In some embodiments, rear guard portion 102a and front guard portion 102b may be separate pieces that are sealed together substantially around the base portion 110. For example, in various embodiments, rear guard portion 102a and front guard portion 102b may be snapped together to create a seal. Further, in some embodiments, rear guard portion 102a and front guard portion 102b may be affixed together with an adhesive, or the pieces can be welded together. In some embodiments, the guard portion 102, including the rear guard portion 102a and front guard portion 102b, may comprise a single piece created by a process of, for example, injection molding. In various embodiments, the guard portion 102 may comprise a plastic material. The guard portion 102 may comprise a substantially transparent material, according to some embodiments. In various embodiments, the guard portion 102 may comprise a material having a low thermal resistivity or a high thermal conductivity, such that the guard portion 102 may easily transfer heat from the surrounding environment to the base portion 110. In some embodiments, the guard portion 102 may have a thermal conductivity of 0.2 W/mΩ·K or more. As shown in FIG. 2G, in some embodiments, a handle member 108 may be coupled to the guard portion 102 by the ends of the handle member 108 being received by, for example, cavities 118 and notches 120 integrated into the rear guard portion 102a. The ends of the handle member 108 may be secured between the rear guard portion 102a and the front guard portion 102b.

As shown in FIG. 2D, in some embodiments, the neck portion 104, the nipple portion 106 and the base portion 110 of a temperature-sensitive pacifier 100 may comprise a single, unitary temperature-sensitive piece 114. In other embodiments, the neck portion 104 and nipple portion 106 may form a single piece that is coupled to the base portion 110. In still other embodiments, the neck portion 104, nipple portion 106 and base portion 110 all may be separate pieces that are coupled together, by, for example, welding or by use of an adhesive. In some embodiments, the base portion 110 of a unitary temperature-sensitive piece 114 may be substantially encapsulated by the guard portion 102 through a process of, for example, injection molding. In some embodiments, the base portion 110, rear guard portion 102a, and front guard portion 102b may have one or more apertures 116, which may align with one another to provide one or more holes through the body of the temperature-sensitive pacifier 100. These holes may provide a safety benefit by serving to prevent a vacuum seal from forming between the pacifier and the face of the infant.

In various embodiments, the nipple portion 106 may be configured to be received by an infant's mouth. As will be understood and appreciated, when placed in an infant's mouth, the nipple portion 106 and neck portion 104 will receive body heat from the inside of the infant's mouth. In some embodiments, the nipple portion 106 may comprise a temperature-sensitive material. In some embodiments, the nipple portion 106 may comprise a first material (e.g., rubber, silicon, plastic, latex, or other suitable material) coated with a temperature-sensitive pigment. Whether the temperature-sensitive material is coated onto a surface of a portion of the pacifier 100 or whether it is integrated into or mixed with the material of the pacifier 100 during manufacturing, the temperature-sensitive material may perform in substantially the same way. In various embodiments, the temperature-sensitive material may be a thermochromic material. As will be understood by one of skill in the art, thermochromism is a property in which the color of the material will change, or transition from one color to another, in response to the addition or removal of thermal energy. For example, a common form of thermochromic manipulation is the inducement of structural changes to molecular geometries. These changes in molecular geometries may cause the materials to reflect a different color of light, or may cause the material to become transparent. Generally, there are two families of thermochromic materials: thermochromic liquid crystals (TLCs) and thermochromic leucodyes. Both TLCs and leucodyes may be produced in the form of a microcapsule suspension (typically referred to as a “slurry” for manufacturing) with particles in the size of 3-5 μm. TLCs and leucodyes may be produced in various configurations or combinations in order to affect a desired color change at a desired temperature. As used herein, a “color change” may be used to refer to a change in color that this perceptible by the human eye, and it may also refer to a change from an opaque state to a transparent state.

Thermochromic liquid crystals (TLCs) may respond to precise changes in temperatures within certain temperature bands. TLCs may be manufactured in the form of microscopic spheres embedded in a plastic or polymer. When the temperature of the TLCs changes, the spacing between the liquid crystals changes, thereby causing the TLCs to reflect light of a different wavelength, which cause a human to perceive a different color. Thus, under different temperature conditions, a surface containing TLCs will appear to have different visual characteristics. For example, depending on the temperature, a surface containing TLCs may appear to change color or change from opaque to transparent (or vice versa). TLCs may also change colors along a color gradient. Thus, in some embodiments, TLCs may cause a portion of the temperature-sensitive pacifier 100 to change to a variety of different colors based on the temperature condition.

Due to the nature of thermochromic materials, a change in color may occur over an interval of temperature change. In various embodiments a “color change” may refer to a change in color of the thermochromic material that is discernible to the human eye. In some embodiments, the color change may occur over a range of temperatures. So, for example, a temperature “set point,” also referred to as an “activation temperature,” may be the temperature at which point the thermochromic material may be configured to begin to change color. The temperature at which the thermochromic material may be configured to have substantially completed a change from a first color to a second color may be referred to as the “end point.” In some embodiments, a color change may be said to be substantially complete when 95% of the desired color change has occurred. In some embodiments, the process of a color change may occur exponentially as the temperature rises from the set point to the end point. So, for example, while a color change process may begin at the temperature set point, and the color change may be marginally perceptible at the set point, the color change process generally comprises an exponential color change as the temperature nears the end point.

TLCs may be generally formulated to change colors anywhere in the range of −22° F. to +248° F. (−30° C. to 120° C.) and may be sensitive enough to change colors in response to temperature changes as small as 0.2° F. Thus, in some embodiments, TLCs may be configured to change color in a manner that is discernible to the human eye in increments of as small as 0.2° F. For example, in one embodiment, a TLC may have a set point of 99.5° F. and an end point of 99.7° F. Accordingly, in this case, as the temperature rises above 99.5° F. the TLC will begin to change from a first color to a second color, and when the temperature reaches 99.7° F., the TLC will have substantially finished changing to a second color. As TLCs change color according to a gradient, they may be configured to change from one color to another in successive increments. For example, according to some embodiments, a temperature-sensitive pacifier having TLCs may be configured to display a first color at 99.5° F., a second color at 99.7° F., a third color at 99.9° F., and so on. It should be understood that the 0.2° F. increment are merely exemplary and that a temperature-sensitive pacifier having TLCs may be configured to change colors in larger increments, and also in increments that vary in magnitude from step to step.

Similarly, thermochormic leucodyes are temperature-sensitive dyes (or inks) that may also change color in response to a change in temperature. As described above, leucodyes may be produced in microcapsules. In some embodiments these microcapsules operate by halochromism. The microcapsules may comprise a dissociable salt and a weak acid. The dissociable salt may be dissolved in a solvent such as dodecanol. If the temperature is below the melting point of the solvent, the color forming components of a polymeric microcapsule may be in contact, creating the appearance of color. If the temperature is above the melting point of the solvent, the color forming components may be separated, thereby no longer producing the appearance of color.

Accordingly, in response to a temperature change, thermochromic leucodyes may become transparent or opaque. In contrast to TLCs, thermochromic leucodyes tend to be less responsive to precise changes in temperature. Leucodyes may exist as a mix of colorless leucodye forms and colored forms, or they may exist as a mix of various leucodyes with different temperature set-points. Leucodyes may be printed on the surface of other materials in the form of microscopic capsules. Leucodyes may be organic chemicals that change color when heat energy causes their molecules to shift between two different structures known as the leuco (colorless) form and non-leuco (colored) forms. These two forms absorb and reflect light differently, thereby producing two different visual appearances. Thus, unlike TLCs which may change to multiple different colors along a color gradient, leucodyes generally provide a more binary visual indication of a temperature condition (i.e., “hot” vs. “cold”) by switching between two binary states. However, in various embodiments, lecuodyes may be “layered” such that the combination of layers produces multiple states. According to some embodiments, “layering” leucodyes may comprise creating a mixture of leucodyes having different colors, set points, and end points. For example, in one embodiment, a portion of a temperature-sensitive pacifier 100 may include a first layer leucodye and a second layer leucodye. For example, the first layer leucodye may be blue in its non-leuco form and have an end point of 99.7° F., whereas the second layer leucodye may be red in its non-leuco form have an end point of 102° F. At room temperature both layers would be in their non-leuco form, and thus the combination of blue and red colors of the two layers may give the portion of the temperature-sensitive pacifier 100 a purple appearance. According to this embodiment, when the temperature increases to 99.7° F., the first leucodye may change to its leuco state and change from blue to colorless (or transparent), thus changing the color of the portion of the temperature-sensitive pacifier from purple to red, thereby providing an indication of a first temperature condition. According to this embodiment, when the temperature increases to 102° F. or beyond, the second layer leucodye may also change to its leuco state, thereby changing the appearance of the portion of the pacifier to clear or transparent and providing an indication of a second temperature condition. So, as the infant's temperature continues to go up, the temperature-sensitive pacifier 100 may change in appearance from purple, to red, to transparent. It should be understood that in various embodiments, portions of a temperature-sensitive pacifier 100 may contain one or more layers of leucodyes that may be configured to create one or more color changes at predefined temperatures.

Organic leucodyes generally may be formulated to change colors anywhere in the range of 23° F. to 140° F. (−5° C. to 60° C.). Similar to TLCs, leucodyes typically can change from a first color to a second color over the course of a change in temperature from a set point to an end point. Organic leucodyes generally can change colors in intervals (i.e., the difference in the set point and the end point) of approximately 5.4° F. (about 3° C.). It should be understood that a wide variety of combinations of colors, set points, and end points may be achieved by combining or layering various leucodyes and thus, embodiments having innumerable combinations that may provide visual indications of a variety of different temperature conditions are contemplated herein.

In some embodiments, the neck portion 104, the nipple portion 106 and the base portion 110 may be made from, or coated with, one or more thermochromic materials, such as, for example, but not limited to, thermochromic liquid crystals or thermochromic leucodyes. Any portion (i.e., neck portion 104, nipple portion 106, and/or base portion 110) that is integrated with a thermochromic material may be configured to change color, lose color, become transparent or become opaque in response to a predetermined change in temperature. It is contemplated that in various embodiments, each of the neck portion 104, nipple portion 106, and base portion 110 may or may not be made from, or coated with, a thermochromic material. Accordingly, in various embodiments, different portions may comprise different thermochromic materials or may comprise no thermochromic materials at all. It should be understood that this disclosure contemplates various different placements, configurations, temperature set points, sensitivities, and color ranges (including transparency and opaqueness) of the thermochromic materials with respect to the neck portion 104, the nipple portion 106 and the base portion 110, including embodiments wherein one or more of the neck portion 104, the nipple portion 106 and the base portion 110 may not include any thermochromic material.

As discussed, in some embodiments, the nipple portion 106 of the pacifier 100 may provide a visual indication of an oral temperature condition. For example, the nipple portion 106 may change or lose color in response to absorbing heat from the infant's mouth. As noted, oral temperature of a human may provide an indication of the body temperature of the human. Thus, in some embodiments, the nipple portion 106 may change or lose color in response to an oral temperature indicative of the infant having a fever. For example, the nipple portion 106 may be configured to change or lose color if the infant's oral temperature changes from below 99.7° F. to 99.7° F. or above. However, it should be understood that portions of the temperature-sensitive pacifier having thermochromic materials may be configured to change or loss color at different temperatures across different embodiments. In some embodiments, the neck portion 104 may change or lose color in the same manner as the nipple portion 106. Accordingly, if a baby is asleep with the temperature-sensitive pacifier 100 in his or her mouth, a parent may inspect the neck portion 104 for a change in visual characteristic by only partially removing the pacifier 100 from the baby's mouth.

In some embodiments, the base portion 110 may provide a visual indication of an environmental temperature condition. For example, the base portion 110 may change or lose color in response to absorbing heat from the external environment surrounding the temperature-sensitive pacifier 100. For example, the base portion 110 may absorb heat from the ambient air surrounding the temperature-sensitive pacifier 100. The temperature of the external environment surrounding the temperature-sensitive pacifier 100 may provide an indication of an external temperature condition that the user of the temperature-sensitive pacifier is being subjected to. Thus, in some embodiments, the base portion 110 may change or lose color in response to an environmental temperature indicative of an environmental temperature condition that poses a risk of heatstroke to an infant. For example, the base portion 110 may be configured to change or lose color if the environmental temperature changes from below 99.7° F. to 99.7° F. or above.

Similarly, in some embodiments, the nipple portion 106 may comprise a thermochromic material configured to change or lose color when placed in an infant's mouth, in response to an oral temperature indicative of the infant having a fever. According to some embodiments, the nipple portion 106 may change or lose color when the oral temperature of the infant is at or above 99.7° F. In some embodiments, the base portion 110 may comprise a thermochromic material configured to change or lose color in response to an environmental temperature indicative of a risk of an infant suffering from heatstroke. According to some embodiments, the base portion 110 may change or lose color when the environmental temperature of the air surrounding the external surface of the pacifier is at or above 99.7° F. In various embodiments, the nipple portion 106 and base portion 110 may change or lose color independently from one another. For example, if the infant has a fever but it is cold outside, the nipple portion 106 may change or lose color when placed in the infant's mouth, but the visual characteristics of the base portion 110 may remain unchanged. Likewise, in another example, if it is very hot outside, but the infant's temperature is within normal ranges, then the base portion 110 may change or lose color but the visual appearance of the nipple portion 106 may remain unchanged when placed in the infant's mouth. According to some embodiments, both the nipple portion 106 and the base portion 110 may be configured to change or lose colors under substantially the same temperature conditions. For example, the existence of a fever and a risk of heat stroke may both occur at temperatures of 99.7° F. or above. Thus, in some embodiments, both the nipple portion 106 and base portion 110 may be configured to change or lose color at 99.7° F.

In various embodiments, the rear guard portion 102a may comprise a transparent material. This feature may allow a person to visually observe an indication of an external temperature condition indicated by the base portion 110 by merely looking through the transparent rear guard portion 102a of the temperature-sensitive pacifier 100 and observing the visual characteristics of the base portion 110. Thus, the temperature-sensitive pacifier 100 may enable a parent to passively monitor whether an external temperature condition exists that could lead to heatstroke simply by looking at an infant's temperature-sensitive pacifier 100. In various embodiments, a person may visually observe an indication of a body temperature condition by looking at the nipple portion 106 and/or neck portion 104 of the temperature-sensitive pacifier after it has been in the infant's mouth for a period of time. In some embodiments, the nipple portion 106 and/or neck portion 104 of the temperature-sensitive pacifier may change or lose color when placed in the mouth of an infant with a fever after approximately 30 seconds. Thus, a parent may passively monitor for an early warning of a potentially dangerous fever condition or risk of heat stroke by simply observing the visual characteristics of the nipple portion 106 and/or neck portion 104 when the temperature-sensitive pacifier is from time to time removed from the infant's mouth.

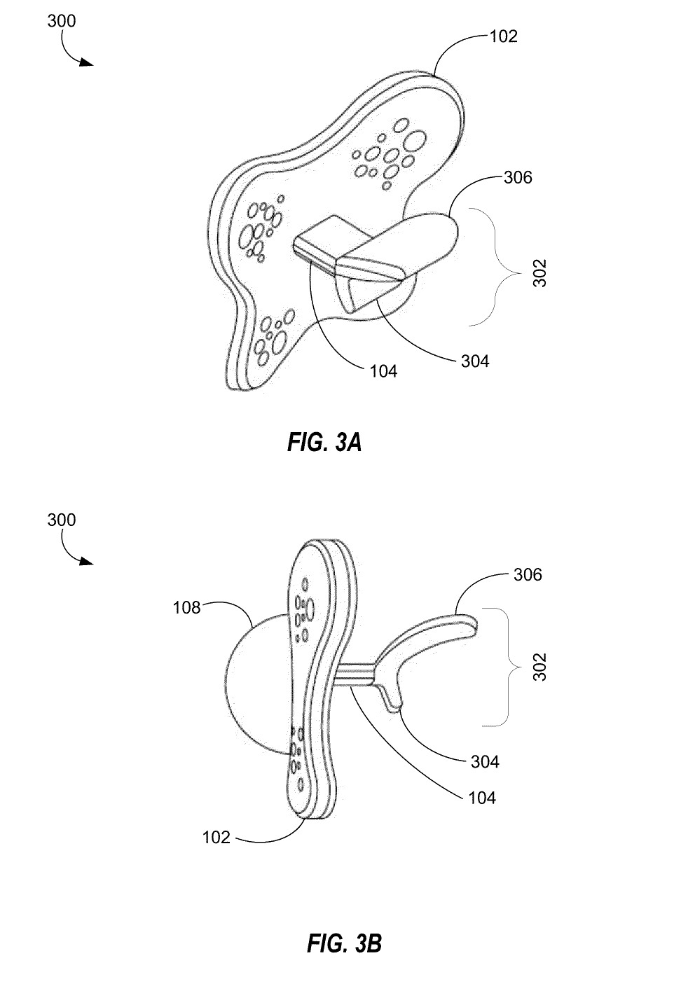

As shown in FIGS. 3A-B, some embodiments of a temperature-sensitive pacifier 300 may provide a branched nipple portion 302 having a lower branch 304 and an upper branch 306. In addition to the early warnings of various temperature conditions provided by the temperature-sensitive pacifier 300, the branched nipple portion 302 may provide additional benefits to the development of an infant. Some pacifier designs have been associated with an increased prevalence of harmful conditions such as malocclusion, anterior open bite, excessive overjet, unilateral posterior crossbite, the proclination of the upper incisors, and a reduced intercanine distance of the upper arch. Further, some pacifier designs have been associated with various skeletal deformations, such as mandibular and maxillary malformations, palatal distension, and an increase of the dolichocephalic angle of the face. Further still, some pacifier designs have been associated with oral musculature deformities, such as incompetent lip and tongue tonus, improper tongue rest position, improper tongue thrust, and anterior tongue interposition during swallowing, which have been associated with the development of various speech pathologies. Accordingly, a pacifier that offers palatal support of the upper arch, proper tongue positioning in the oral cavity, and minimized maxillary-mandibular proximity to decrease dental deformities would be advantageous. Some embodiments of a branched temperature-sensitive pacifier 300 may provide such benefits to assist in the healthy development of an infant.

According to some embodiments, the lower branch 304 may comprise a member that extends downwards away from the neck portion 104. In some embodiments, the upper branch 306 may extend away from the neck portion 104 in a substantially horizontal and vertical fashion. In some embodiments, the lower branch 304 may be less than half the length of the upper branch 306. In some embodiments, the lower branch 304 may have substantially the same width as the upper branch 306.

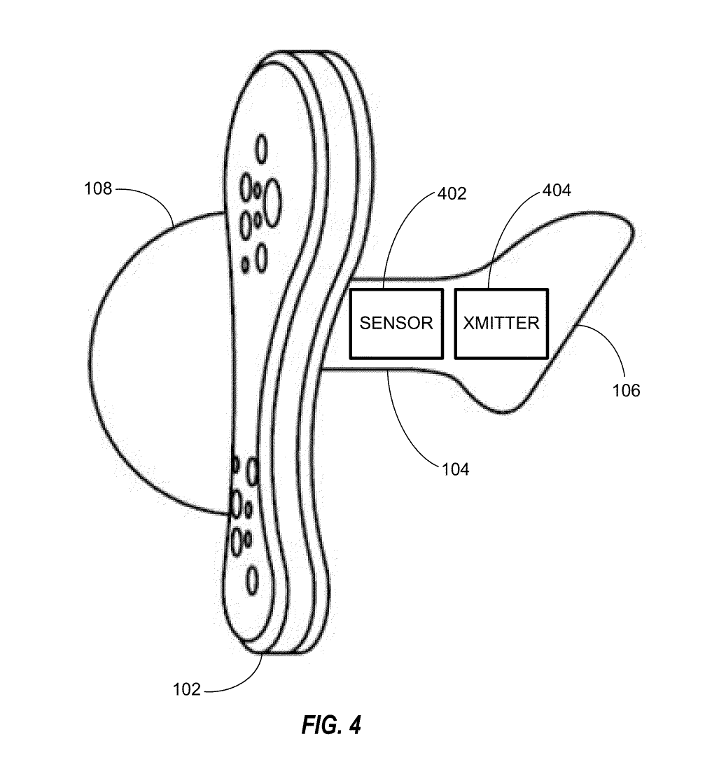

As shown in FIG. 4, some embodiments of a temperature-sensitive pacifier 400 may be equipped with at least one sensor 402 and a transmitter 404. In some embodiments, the at least one sensor 402 and the transmitter 404 may be partially or wholly encompassed within the nipple portion 106, the neck portion 104, the base portion 110, or within the guard portion 102. The at least one sensor 402 may be configured to receive information from one or more of internal portions or external surfaces of the nipple portion 106, the neck portion 104, or the base portion 110. In various embodiments, the at least one sensor may be operable to detect temperature, sound, acceleration, location, pressure, movement, or other data. For example, in some embodiments, the at least one sensor may be configured to detect the temperature of the nipple portion 106, which is generally indicative of the child's temperature. In some embodiments, the sensor may be configured to detect the temperature of a location external to the pacifier, for example, the temperature of the ambient air surrounding the guard portion 102. According to some embodiments, the at least one sensor may comprise a pressure sensor that may be embedded within the nipple portion 106 to detect the suction pressure exerted by an infant utilizing the pacifier.

The transmitter 404 of a temperature-sensitive pacifier 400 may be in electronic communication with the at least one sensor 402. In various embodiments, the transmitter 404 may receive sensor data collected from the at least one sensor 402. The transmitter 404 may wirelessly transmit the sensor data, or data representative of the sensor data, to another device. For example, in some embodiments, the transmitter 404 may transmit data to a mobile computing device such as a smartphone or tablet. A mobile computing device may contain a processor for processing computer instructions and a display interface that acts as a communication interface and provides functions for rendering video, graphics, images, and texts on the display. A mobile computing device may also be referred to as a mobile station (MS), terminal, cellular phone, cellular handset, personal digital assistant (PDA), wireless phone, organizer, handheld computer, desktop computer, laptop computer, set-top box, television, appliance, game device, medical device or some other like terminology. In some embodiments, the mobile computing device may be configured to provide alerts in response to the received sensor data. So, for example, if the sensor 402 detects that the child has a temperature, the mobile computing device may provide an alert to the child's parent. In some embodiments, the sensor data may be transmitted to a central computer where it may be remotely accessible. For example, a parent may remotely monitor the temperature of the nipple portion 106 and/or base portion 110 by accessing a website on their home computer. The transmitter 404 may utilize any wireless communication method or protocol known, such as for example, Bluetooth or WiFi, or any such wireless communication method devised in the future.

While certain embodiments of the disclosed technology have been described in connection with what is presently considered to be the most practical embodiments, it is to be understood that the disclosed technology is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the disclosed technology, including the best mode, and also to enable any person skilled in the art to practice certain embodiments of the disclosed technology, including making and using any devices or systems and performing any incorporated methods. The patentable scope of certain embodiments of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A temperature-sensitive pacifier comprising:
a nipple portion, the nipple portion comprising a first thermochromic material configured to provide a visual indication of a body temperature condition;
a neck portion coupled to the nipple portion;
a base portion coupled to the neck portion, the base portion comprising a second thermochromic material configured to provide a visual indication of an environmental temperature condition; and
a guard portion substantially encapsulating the base portion, the guard portion comprising a substantially transparent material.

2. The temperature-sensitive pacifier of claim 1, wherein the nipple portion provides a visual indication of a body temperature condition in response to absorbing heat from a mouth of a user.

3. The temperature-sensitive pacifier of claim 1, wherein the base portion provides a visual indication of an environmental temperature condition in response to absorbing heat from a surrounding environment.

4. The temperature-sensitive pacifier of claim 1, wherein the nipple portion, neck portion, and base portion comprise a single member.

5. The temperature-sensitive pacifier of claim 4, wherein the single member is partially encapsulated by the guard portion through a process of injection molding.

6. The temperature-sensitive pacifier of claim 1, wherein the visual indication of a body temperature condition comprises the nipple portion transitioning from a first color to a second color.

7. The temperature-sensitive pacifier of claim 6, wherein the second color is transparent.

8. The temperature-sensitive pacifier of claim 6, wherein the change from the first color to the second color indicates that the user has a body temperature indicative of a fever.

9. The temperature-sensitive pacifier of claim 6, wherein the change from the first color to the second color is substantially complete at an oral temperature of 99.7 degrees Fahrenheit.

10. The temperature-sensitive pacifier of claim 1, wherein the visual indication of the environmental condition comprises the base portion transitioning from a first color to a second color.

11. The temperature-sensitive pacifier of claim 10, wherein the second color is transparent.

12. The temperature-sensitive pacifier of claim 10, wherein the change from the first color to the second color indicates that surrounding environment has a temperature that is indicative of a risk of heat stroke.

13. The temperature-sensitive pacifier of claim 10, wherein the change from the first color to the second color is substantially complete at a temperature of 99.7 degrees Fahrenheit.

14. The temperature-sensitive pacifier of claim 1, further comprising a handle member coupled to the guard portion.

15. The temperature-sensitive pacifier of claim 1, further comprising:
a processor;
at least one sensor in communication with the processor, the at least one sensor configured to measure at least one temperature of the nipple portion or of the base portion; and
a wireless transmitter in communication with the processor, the wireless transmitter configured to wirelessly transmit data indicative of the at least one temperature to a computing device.

16. The temperature-sensitive pacifier of claim 1, wherein the guard portion comprises a material having a thermal conductivity of at least 0.2 W/mΩ·K.

17. The temperature-sensitive pacifier of claim 1, wherein the first thermochromic material and/or the second thermochromic material comprise thermochromic liquid crystals.

18. The temperature-sensitive pacifier of claim 1, wherein the first thermochromic material and/or the second thermochromic material comprise leucodyes.

19. The temperature-sensitive pacifier of claim 18, wherein the leucodyes comprise two or more leucodye layers, each leucodye layer having a different color and a different set point.

20. The temperature-sensitive pacifier of claim 1, wherein the nipple portion comprises an upper branch member and a lower branch member.
